(19) United States
(12) Patent Application Publication
(10) Pub. No.: US 2023/0268051 A1
(43) Pub. Date: Aug. 24, 2023

(54) ARTIFICALLY INTELLIGENT MEDICAL PROCEDURE ASSESSMENT AND INTERVENTION SYSTEM

(71) Applicant: Purdue Research Foundation, West Lafayette, IN (US)

(72) Inventors: Juan Pablo Wachs, West Lafayette, IN (US); Edgar Javier Rojas-Munoz, West Lafayette, IN (US)

(73) Assignee: Purdue Research Foundation, West Lafayette, IN (US)

(21) Appl. No.: 18/012,529

(22) PCT Filed: Jun. 24, 2021
(86) PCT No.: PCT/US2021/038968

§ 371 Date: Dec. 22, 2022

Related U.S. Application Data
(60) Provisional application No. 63/046,298, filed on Jun. 30, 2020.
(60) Provisional application No. 63/046,246, filed on Jun. 30, 2020.

(51) Int. Cl. G16H 20/40; G06N 3/044; G06N 3/08; G16H 30/40; G16H 70/20; G16H 40/67

(52) U.S. Cl. CPC G16H 20/40; G06N 3/044; G06N 3/08; G16H 30/40; G16H 70/20; G16H 40/67

(57) ABSTRACT

A system and methods for artificially intelligent medical procedure assessment and intervention are provided. A system may acquire image data from a camera targeting a location where healthcare is administered. The system may receive sensor data from a sensor attached to a care provider during performance of a procedure. The system may generate gesture features based on the sensor data. The system may generate image features based on the image data. The system may determine, based on the image features and a machine learning model, a step identifier for a step in a multi-step surgical procedure. The system may access text descriptive of an instruction to perform the step. The system may display the text on a display accessible to the care provider. The system may determine, based on the gesture information and a second machine learning model, a performance metric that measures performance of the care provider.

### CROSS REFERENCE TO RELATED APPLICATIONS

This application claims the benefit of U.S. Provisional Application No. 63/046,246 filed Jun. 30, 2020 and U.S. Provisional Application No. 63/046,298 filed Jun. 30, 2020, the entirety of each of these applications is hereby incorporated by reference.

### GOVERNMENT RIGHTS

This invention was made with government support under W81XWH-14-1-0042 awarded by the ARMY/MRMC. The government has certain rights in the invention.

### BACKGROUND

Telementoring surgeons as they perform surgery can be essential in the treatment of patients. Nonetheless, expert mentors are often unavailable to provide trainees and caregivers with real-time medical guidance. In addition, real time communications with expert mentors is subject to the reliability of computer networks and distributed systems.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates a first example of a system 100.

FIG. 2 illustrates a flow diagram for logic of a system involving step identification for a multi-step medical procedure.

FIG. 3 illustrates a flow diagram for logic of a system involving artificially assisted intervention in a multistep medical procedure.

FIG. 4 illustrates a second example of a system.

FIG. 5 Illustrates a flow diagram for logic of a system involving telemonitoring intervention in a multi-step medical procedure.

FIG. 6 illustrates a flow diagram for logic of a system involving failover and artificially assisted intervention.

FIG. 7 illustrates a third example of a system.

### DETAILED DESCRIPTION

Telementoring may provide general surgeons or trainees with remote supervision when no expert specialist is available on-site. Disruptions in connectivity and computer performance can detrimentally affect the performance of medical care in both live and training settings. Moreover telemonitoring expertise may be limited or not available at all times. Accordingly, systems and methods for artificially intelligent medical procedure assessment, intervention, and failover are provided. By way of introductory example, system may acquire image data from a camera targeting a location where healthcare is administered. The system may receive sensor data from a sensor attached to a care provider during performance of a multi-step medical procedure. The system may generate gesture features based on the sensor data. The system may generate image features based on the image data. The system may determine, based on the image features and a machine learning model, a step identifier for a step in a multi-step surgical procedure. The system may access natural language text descriptive of an instruction to perform the step. The system may display the natural language text associated with the step on a display accessible to the care provider. The system may determine, based on the gesture information and a second machine learning model, a performance metric that measures performance of the care provider.

In another example, the system may monitor a network communication between a care provider proximate to first geographic location where care is administered and a remote user at a second geographic location. Concurrent with the communication session, the system may receive real-time image data of a medical procedure administered at the first location, generate image features based on the image data, and determine, based on the image features and a machine learning model, a step identifier for a step in a multi-step surgical procedure. In response to compromised communication between the care provider and the remote user, the system may access instructional information descriptive of the step. The instructional information may include a combination of natural language text and speech. They system may display the instructional information on a display at the first location. Additional examples and technical advantages are made evident in the system and methods described herein.

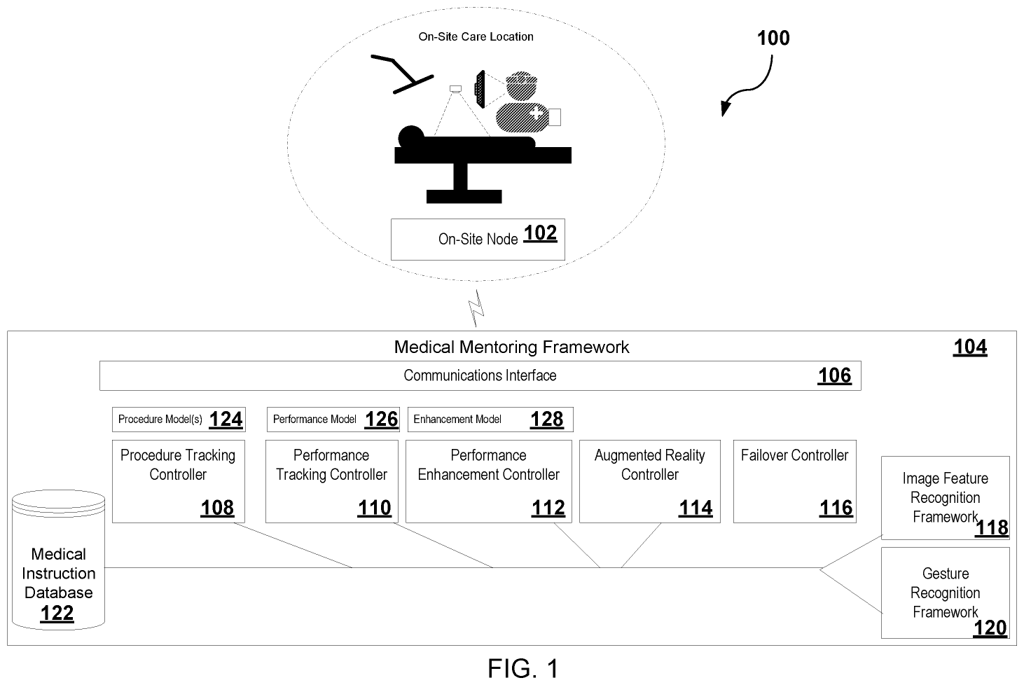

FIG. 1 illustrates a first example of a system 100. Among other aspects, the system 100 may provide artificially intelligent scoring, assisted intervention, and assisted failover during a medical procedure performed by a care provider. As described herein, a care provider refers to a person that performs physical actions to carry out a procedure for administering medical care. The care provider may be, for example, a surgeon, nurse, emergency responder, a medical student, or any person administering medical care. An onsite location refers to a geographic location where care is physically administered. For example, the on-site location may be an operating room, a hospital, an incident location, an ambulance, a medical training center, or any other geographic location where care is administered.

The system 100 may include an on-site node 102 at the on-site location. The on-site node 102 may include, or communicate with, a plurality of devices that capture audio, video, and interactive operations at the on-site care location. For example, the devices at the on-site location may include an audio capture device (i.e. a microphone), a display device (i.e. tablet or headset), an audio playback device (i.e. speaker), computer peripherals, etc. One or more of the on-site devices may be included in an augmented reality device, such as a headset. Alternatively, or in addition, the onsite devices may be integrated with the on-site location. For example, the display device may be mounted over a surface or area where medical care, such as surgery, is administered. As defined herein, the devices located on-site are may be collectively referred to as “on-site devices”.

In some examples, at least of one of the on-site devices may include an sensor attachable coupled to the care provider. For example, the sensor may be included in or secured by an armband that connects to the arm, wrist, or hand of the care provider. The sensor may collect various types of information that measures movement by the care provider. For example, the sensor may generate a measurement of directional movement, rotation, vibration, position, or a combination thereof. Alternatively or in addition, the sensor may measure electrical activity of the muscles of the care provider. In an example, the sensor may be the Myo armband and generate electromyography (EMG) measurements.

The system 100 may include an artificially intelligent medical mentoring framework 104 (hereinafter referred to a mentoring framework). The mentoring framework 104 may include a communications interface 106, a procedure tracking controller 108, a performance tracking controller 110, a performance enhancement controller 112, an augmented reality controller 114, a failover controller 116, an image recognition framework 118, a gesture recognition framework 120, and/or a medical instruction database 122.

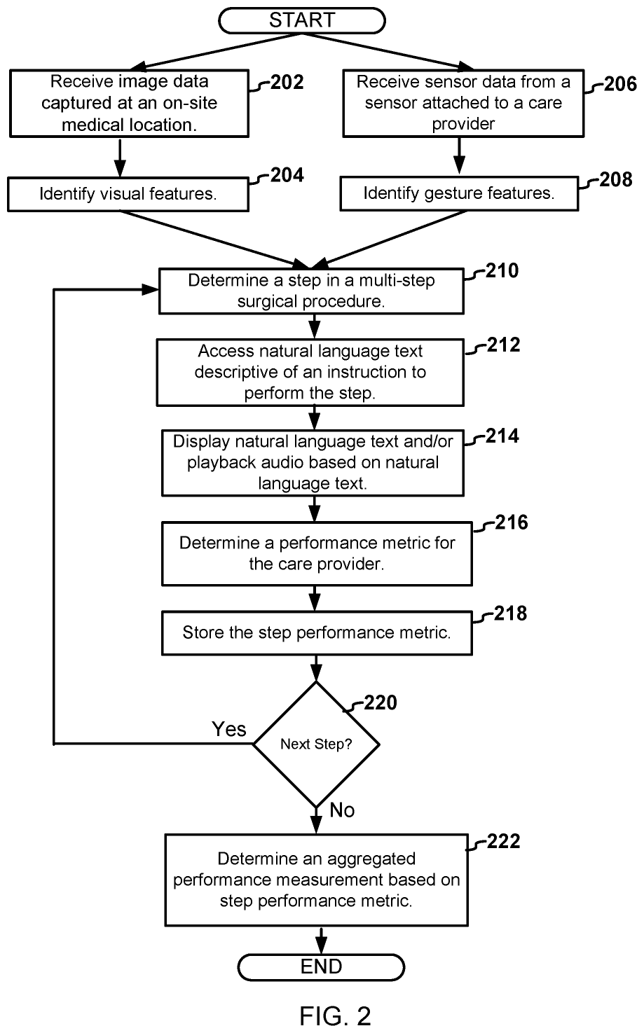

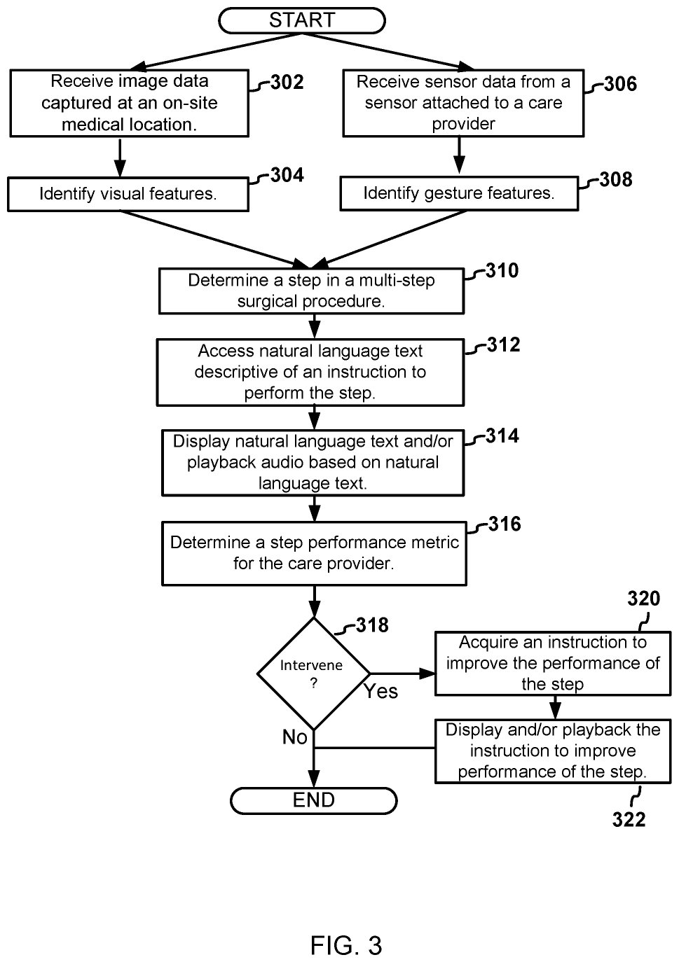

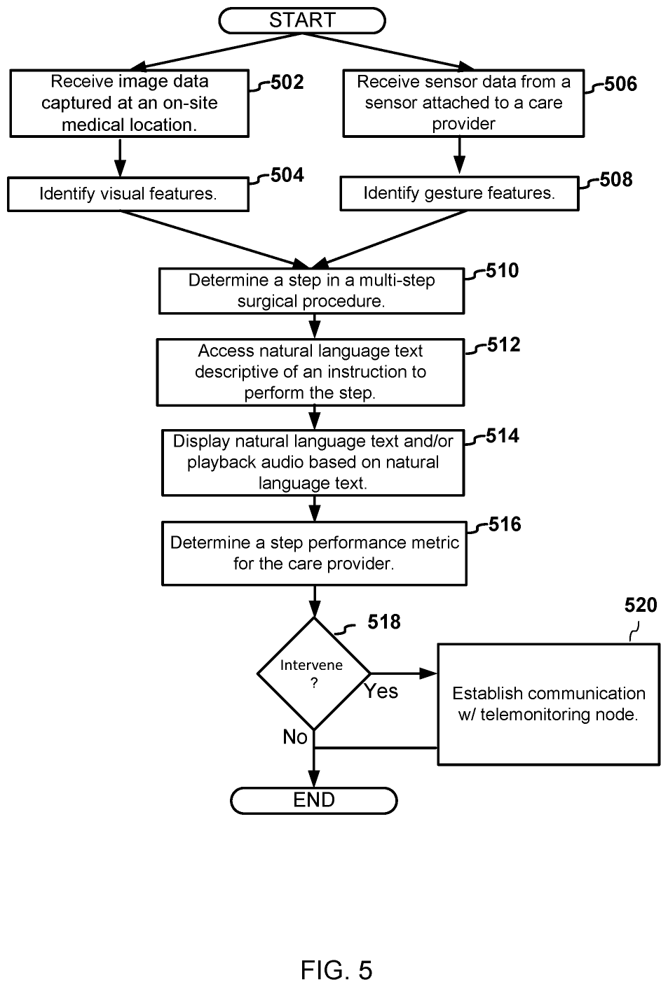

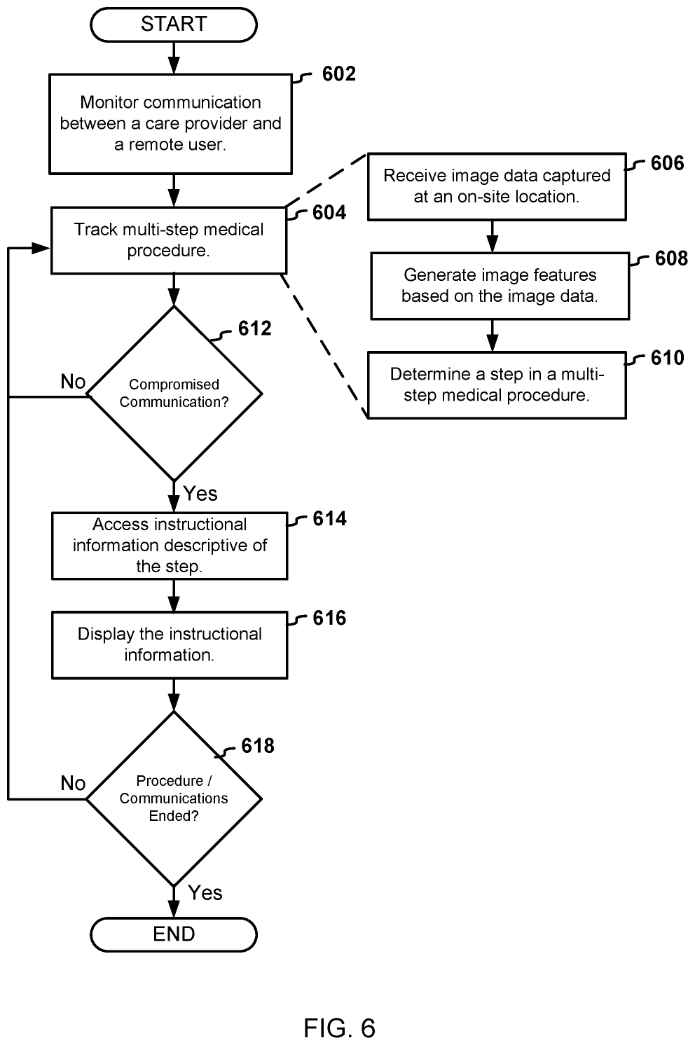

FIGS. 2-8 provide a description of these components and examples of their interactions. As discussed in reference to FIG. 2, the mentoring framework may generate aggregated performance measurements based on the care administered by a professional. As discussed in reference to FIG. 3 and FIG. 5, the mentoring framework may intervene during performance of care causing artificially intelligent mentoring and/or telemonitoring mentoring. As discussed in FIG. 6, the mentoring framework may also provided assisted artificially intelligent intervention when communications between a live mentor and care provider becomes compromised.

The medical instruction database 122 contains may include color images, sensor data, and text descriptions of instructions to perform surgical procedures performance metrics, and/or enhancement suggestions. Associations between the data types may be formed to create data sets. The datasets may be divided into training and testing sets to accommodate machine learning model training. By way of example, the medical instruction database and machine learning model training may be performed as described in Rojas-Munoz, E., et al., “DAISI: Database for AI Surgical Instruction”, DeepAI, 22 Mar. 2020, which is incorporated by reference herein.

The system 100 may include one or more machine learning model trained on the aforementioned medical information. For example, the procedure tracking controller may access one or more procedure model 124 to predicts steps based image data and/or sensor data. The procedure controller 108 may receive images and/or sensor data from medical procedures as input, and predicts an instruction associated with it. To generate text information from images, an encoder-decoder DL approach using a Convolutional Neural Network (CNN) and a Recursive Neural Network (RNN) was adopted. The CNN extracts and encodes visual features from the input images, and the RNN decodes these visual features into text descriptions.

The system may include additional and alternative machine learning models. For example, the system may include a performance model 126 and/or an enhancement model 128. Additional description of these models is provided in FIGS. 2-3 below. It should be appreciated that, in some examples, the performance, enhancement, and procedure models, or a combination there of, may be combined in to a single model in which associations between gesture features, image features, step identifiers, performance scores, and other information acquired by derived is used to form predictions according to machine learning concepts.

FIG. 2 illustrates a flow diagram for logic of the system 100 involving step identification for a multi-step medical procedure. Reference to FIG. 1 is made throughout the following discussion of FIG. 2. The communications interface 106 may receive image data captured at an on-site location (202). For example, the camera at the onsite location may capture image data before and/or during a medical procedure. The image data 106 may include images (pictures), video data, and/or video frames. It should be appreciated that the image data may be received in real time, or near real time, as a procedure is conducted (or about to be conducted) at the on-site location.

The image feature recognition framework 118 may identify visual features in the image data captured at the on-site location (204). For example, the image feature recognition framework 118 may access a machine learning model that generates image features based on visual features. The machine learning model may include, for example, a convolutional neural network (CNN). The image feature recognition framework may encode the visual features with the (CNN) to generate the image features. The resultant image features may include edges, corners, regions of interest, types, or other relevant image features.

The communication interface 106 may receive sensor data from the sensor attached to the care provider (206). The sensor may generate the sensor data during the multi-step procedure.

The gesture recognition framework may identify gesture features based on the sensor data (208). For example, the gesture recognition framework 120 may use the sensor data as input to a machine learning model to classify the sensor data. The machine learning model may include, for example, an electromyography-based model where electrical signals produced by muscles are classified into gesture information. The gesture features may include a classification of human movement from body parts used by the care provider to perform a medical procedure. For example, the gesture recognition framework may identify particular movements of the arm and/or hand based on the sensor data.

The procedure tracking controller may determine a step in a multi-step surgical procedure (210). The procedure tracking controller 108 may access the procedure tracking model to generate inferences as to the current step in a multi-step medical procedure. The procedure tracking model may include, for example, a procedure model trained to identify steps and/or instructional information based on information included in the medical instruction database. By way of example, the procedure model may include a recurrent neural network (RNN). The procedure tracking controller may decode the image features with the RNN. The resultant output of the RNN may include one or more step identifiers representative of a step in a multi-step medical procedure.

In some examples, the medical instruction database 122 may associate the step identifier with various step information relevant to performing the medical step. For example, the step information may include, for example, text and speech describing how to perform the step, a video of the medical procedure, and/or other relevant information.

The augmented reality controller 114 may access natural language text descriptive of an instruction to perform the step (212). The natural language text associated may be associated with a step identifier for the current step in the multi-step medical procedure. The procedure tracking controller may provide the augmented reality controller with information related to the latest step in a multi-step medical procedure, such as the step identifier.

The augmented reality controller 114 may cause the natural language text to be displayed or played back (via audio) at the on-site location (214). For example, the augmented reality controller may communicate the instructional information to the on-site node or directly to the display device and/or audio playback device. In either case, the on-site care provider may view/hear the instructional information for assistance with the multi-step medical procedure.

In some examples, the augmented reality controller 114 may receive an identifier for the current step. The augmented reality controller may access the instructional information from the medical instruction database. The augmented reality controller may convey the instructional information to one or more on-site devices.

By way of examples, the augmented reality controller 114 may access natural language text associated with the current step in the multi-step process. The natural language text may explain how to perform the step. The augmented reality controller 114 may cause the natural language text to be displayed by the display device. Alternatively or in addition, the augmented reality controller 114 may perform text-to-speech conversion of the natural language text and then cause the result audio to be played back by the on-site audio playback device. In some examples, the augmented reality controller may access images or video previously associated with the step identifier and send the images or video to the on-site device(s).

The performance tracking controller 110 may determine a performance metric for the care provider. For example, the performance tracking controller may access a performance model 126 to determine the performance metric. The performance model 126 may include a machine learning model previously trained with inputs such as step identifiers, gesture features, and scoring information. The input may have various formats depending on the type of machine learning model used. In an example, the machine learning model may be a multi-module model where, during runtime, the gesture information and step information may be combined into an input vector. Based on the input vector, the multi-module model may infer an output performance metric. In other examples, the performance scoring model may include a rule-based model.

The performance tracking controller 110 may store the step performance metric (218). The procedure tracking controller 110 may determine whether there is an additional step (220). In response to there being additional steps, the mentoring framework may generate performance metrics for each step, as exemplified in operations 210-220.

In response to no additional steps, the performance tracking controller 110 may determine an aggregated performance measurement based on the step performance measurements (222). The aggregated performance measurement may include a value derived from the performance measurements of multiple steps. In some examples, thus, may represent a performance metric of the entire procedure or a multiple-step portion of the procedure. In some examples, the aggregated performance measurement may include an average or a weighted average of the procedure performance measurement. For example, each step in the multi-step procedure may be assigned a corresponding weight. The weight may be applied to the measured performance metric for the step. The weighted sores may be averaged to determine the aggregated performance metrics.

FIG. 3 illustrates a flow diagram for logic of the system 100 involving artificially assisted intervention in a multistep medical procedure. Reference to FIG. 1 is made throughout the following discussion of FIG. 3. Operations 302-316 of the logic illustrated in FIG. 3 follow operations 202-216 described in reference to FIG. 2 above.

In some examples, the mentoring framework 104 may intervene when the care provider's performance falls below a satisfactory level. For example, the performance enhancement controller 112 may determine whether an intervention criterion is satisfied (318). The intervention criterion may include logic that compares the step performance metric to one or more predetermined value to determine whether intervention should be triggered. For example, the intervention criteria may include a performance metric threshold or range. Intervention may be triggered, for example, when the step performance metric is greater than or less than the performance metric threshold (or outside of the range).

In some examples, the step performance metric may include multiple performance metrics that assess quality of step performance in multiple categories. In such examples, there may be a separate intervention criterion for each category.

In response to satisfaction of the intervention criteria (318, yes), the performance enhancement controller 112 may acquire an enhancement instruction to improve performance of the step (320). The enhancement instruction may include audio, video, or text information that instructs how to improve the performance of the care provider. For example, the enhancement instruction may include natural language text that may be displayed or converted into audio for the care provider to hear.

To acquire the enhancement instruction, the performance enhancement controller 112 may determine an enhancement instruction identifier associated with the intervention criteria. In some examples, the performance enhancement controller 112 may access an enhancement model 128. The enhancement model 128 may include a machine learning model previously trained with features such as the step identifier, gesture features, step performance metrics, instruction identifier or a combination thereof. During runtime, the performance enhancement controller may combine the step identifier, gesture features, and/or step performance metrics into a vector that is used with the enhancement model 128 to identify an enhancement instruction identifier. The enhancement instruction identifier may be associated with instruction information in the medical instruction database.

The augmented reality controller 114 may display or playback the enhancement instruction so that the care provider can improve performance of the step (322). In some examples, the enhancement instruction may be conveyed, via audio playback or video display, in real time while the care provider is performing the step. The augmented reality controller 114 may stop conveying the instruction and/or convey a confirmation that the care provider has corrected his/her performance when intervention is no longer necessary.

In some examples, the logic described in reference to FIG. 3 may repeat operations 310-322 for each step in a multi-step medical procedure.

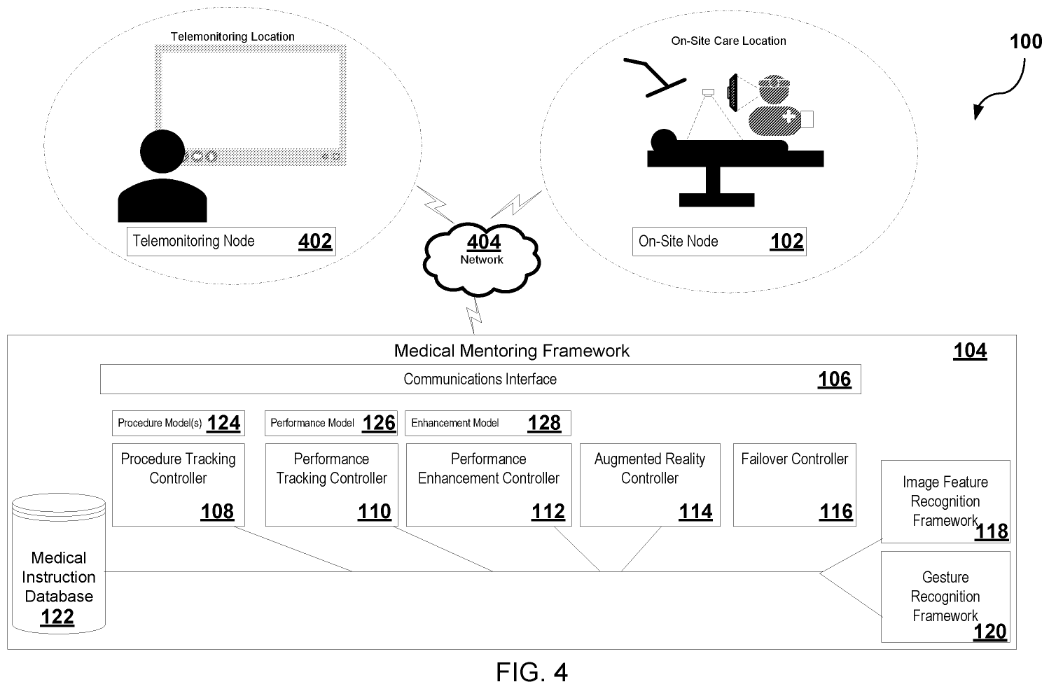

FIG. 4 illustrates a second example of the system 100. The system may include telemonitoring node 402. The telemonitoring node 402 may include, or communicate with, a plurality of devices located at a telemonitoring location remote from the onsite care location. For example, the telemonitoring devices may include an audio capture device (i.e. a microphone), a display device (i.e. a computer screen or head mounted display), an audio playback device (i.e. speaker), computer peripherals, etc. As defined herein, the devices located at the telemonitoring location may be collectively referred to as “telemonitoring devices”.

The telemonitoring node 402, or other telemonitoring devices, and the on-site node 102, or other on-site devices, may communicate over a communications network 404 to exchange information in real-time or near-real time during a medical procedure. Thus, information captured at the on-site location may be transmitted to the telemonitoring node 402, and/or other telemonitoring devices, so the telemonitoring user can view/hear important information related to a medical procedure. Information captured at the telemonitoring location may be transmitted over the communications network to the on-site node 102, and/or other on-site devices, so that the care provider may receive instruction/feedback from the telemonitoring user. Thus, the on-site provider may view/listen to the instructional information with the on-site devices at his/her disposal.

A telemonitoring user at a telemonitoring location may provide instructive information to the care provider. The telemonitoring user may be a person, such as a mentor or assistant, who provides instructive information to the care provider for performing a medical procedure. The instructive information may include, for example, audio, video, text, or other information provided by the telemonitoring user. The telemonitoring location may refer to a location that is physically separate and remote from the on-site care location. In other words, the telemonitoring location is a location where in-person communications are not possible unless performed electronically.

During the course of communications between the telemonitoring location and the on-site care location, network communications may become compromised. Compromised communication refers to a condition where electronic communications between the telemonitoring location and the on-site location are no longer possible or become measurably degraded (i.e. latency, throughput, or some other quantifiable network quality measurement fall below an unacceptable level or outside of an acceptable range).

The artificially intelligent medical mentoring framework 106 may intervene when compromised communications between the telemonitoring location and on-site care location arise.

FIG. 5 illustrates a flow diagram for logic of the system 100 involving telemonitoring intervention in a multi-step medical procedure. Reference to FIG. 4 is made throughout the following discussion of FIG. 5. Operations 502-518 of the logic illustrated in FIG. 3 follow operations 202-218 described in reference to FIG. 2 above.

In this example, intervention may occur in response to the performance metric being less than a satisfactory level (518, Yes). Accordingly, the system may establish communication with the telemonitoring node 402 (520). The telemonitoring node may receive the performance metric, the step identification information, step description, and other information captured sensors at the on-site care location or available in the medical instruction database. A medical expert may provide guidance via video and audio to the surgeon at the on-site care location.

FIG. 6 illustrates a flow diagram for logic of the system 100 involving failover and artificially assisted intervention. Reference to FIG. 4 is made throughout the following discussion of FIG. 6. The failover controller 116 may monitor communications between the on-site device(s) and the telemonitoring device(s) (602). The communications may occur via one or more communications protocols, connections, and/or channels. Alternatively or in addition, the failover controller 116 may monitor information exchanged over one or more layers of a network communication stack. By way of example, a telemonitoring session may be established between the on-site node 102 and the telemonitoring node 402. The failover controller 116 may intercept or receive information exchanged that falls within the scope of the telemonitoring session. Alternatively, or in addition, the failover controller may establish the telemonitoring session between the on-site node 102 and the medical mentoring framework 104. For example, the on-site node may communicate with the mentoring framework 104 to receive medical guidance.

The communications interface 106 may receive image data captured at an on-site medical location (604). For example, the camera at the onsite location may capture image data before and/or during a medical procedure. The image data may be received by both the telemonitoring node 402 and the communications interface 106. Alternatively, the image data may be relayed to the telemonitoring node 402 via the communications interface 106. The communications interface may provide the image data to a communications channel accessible to other components of the medical mentor and/or store the data in a memory or repository for further analysis.

The procedure tracking controller 108 may track the multi-step medical procedure (604). The tracking may involve multiple operations, such as operations 606-610 illustrated in FIG. 6.

During the tracking, the communications interface 106 may receive image data captured at the on-site location (606). The image data may include images (pictures), video data, and/or video frames. It should be appreciated that the image data may be received in real time, or near real time, as a procedure is conducted (or about to be conducted) at the on-site location.

The image feature recognition framework 118 may identify visual features in the image data captured at the on-site location. For example, image feature recognition framework 118 may access a machine learning model that generates image features based visual features. The machine learning model may include, for example, a convolutional neural network (CNN). The image feature recognition framework 118 may encode the visual features with the (CNN) to generate the image features. The resultant image features may include edges, corners, regions of interest, types, or other relevant image features.

The procedure tracking controller 108 may determine a step in a multi-step medical procedure. For example, the procedure tracking controller 108 may receive the image features. The procedure tracking controller 108 may access one or more procedure models 124 to generate inferences as to the current step in a multi-step medical procedure. The procedure model(s) 124 may include, for example, a machine learning model trained to identify steps and/or instructional information based on information included in the medical instruction database. By way of example, the procedure tracking model may include a recurrent neural network (RNN). The procedure controller framework may decode the image features with the RNN. The resultant output of the RNN may include one or more step identifiers representative of a step in a multi-step medical procedure.

In some examples, the medical instruction database may associate the step identifier with various step information relevant to performing the medical step. For example, the step information may include, for example, text describing how to perform the step, a video of the medical procedure, and/or other relevant information.

It should be appreciated that operations 606-610 may occur concurrent with the performance of the multi-step medical procedure and/or communications between the on-site location and the telemonitoring location. Thus, the mentor framework 104 may have information relevant to the latest step in a multi-step medical procedure ready to be accessed during the medical procedure.

The failover controller 116 may determine whether communications between the online location and the telemonitoring location have become compromised (612).

For example, the failover controller 116 may measure communication quality between the on-site device(s) and the telemonitoring device(s). The communications quality measurement may include a metric that quantifies network quality, such as throughput, latency, jitter, error rate, or other relevant network performance metrics.

The failover controller 116 may have a failover criterion. The failover criteria may include a predetermined logic for evaluating the communications quality measurement. For example, the failover criteria may include “Failover if network_performance_measurement<Min_threshold.”

In response to compromised communications (612, Yes), the augmented reality controller 114 may access instructional information associated with the step identifier for the current step in the multi-step medical procedure. For example, when the failover criteria are satisfied, the procedure tracking controller may provide the augmented reality controller 114 with information related to the latest step in a multi-step medical procedure. In response to normal communication (612, No), the communications interface may begin or resume the communication between the on-site provider and the medical mentor.

After detection of compromised communications, the augmented reality controller 114 may cause the instructional information to be displayed or played back at the on-site location (616). For example, the augmented reality controller 114 may communicate the instructional information to the on-site node or directly to the display device and/or playback device. In either case, the on-site care provider may view/hear the instructional information for assistance with the multi-step medical procedure, even if there is compromised communication with the telemonitoring user.

In some examples, the augmented reality controller 114 may receive an identifier for the current step. The augmented reality controller 114 may access the instructional information from the medical instruction database. The augmented reality controller 114 may convey the instructional information to one or more on-site devices.

By way of examples, the augmented reality controller 114 may access natural language text associated with the current step in the multi-step process. The natural language text may explain how to perform the step. The augmented reality controller 114 may cause the natural language text to be displayed by the display device. Alternatively or in addition, the augmented reality controller 114 may perform text to speech conversion of the natural language text and then cause the result audio to be played back by the on-site audio playback device. In some examples, the augmented reality controller 114 may access images or video previously associated with the step identifier and send the images or video to the on-site device(s).

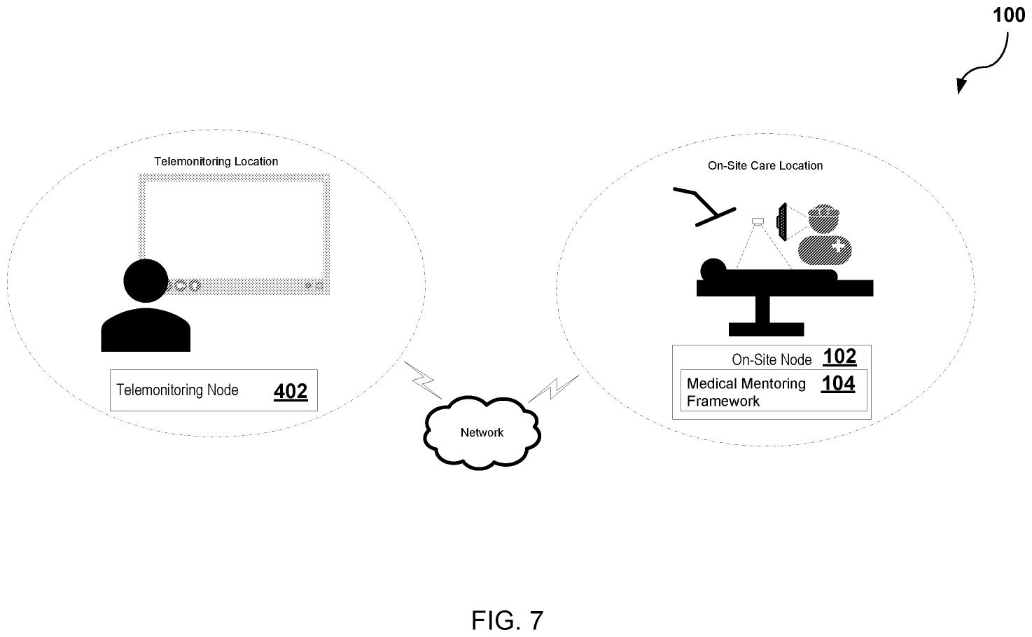

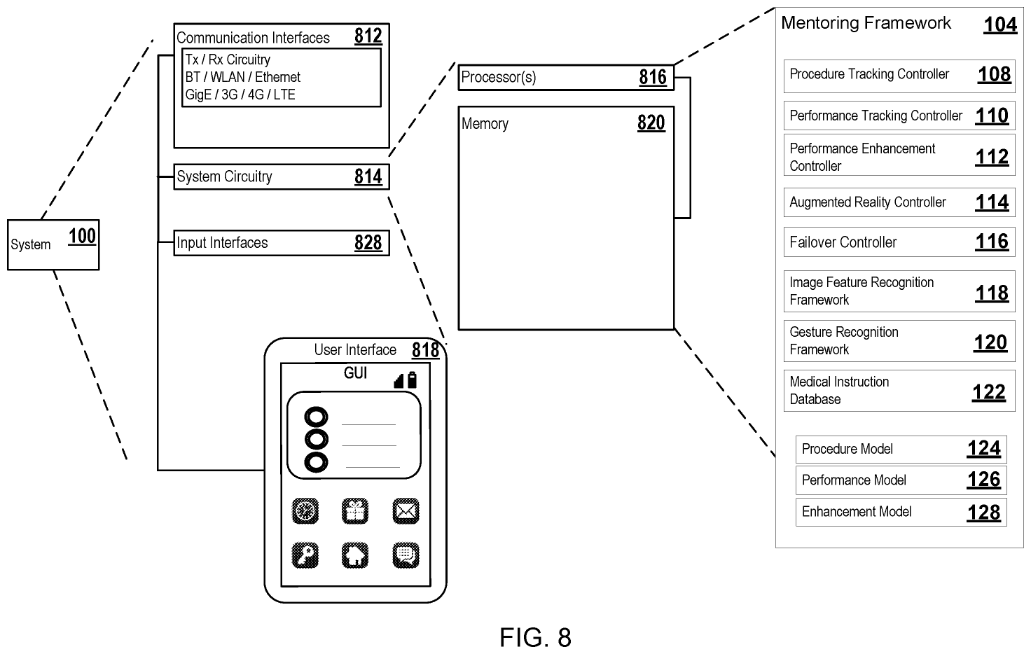

FIG. 7 illustrates a third example of the system 100. The mentor framework 104 may be proximate to the on-site care location. For example the medical mentoring framework 104 may be included or executable on the on-site node. In other examples, the medical mentoring framework 104 may be accessible via a local area network. In yet other examples, some components of the mentoring framework 104 may be executable by a host cloud provider while others are located proximate to the on-site care location.

The system 100 may be implemented with additional, different, or fewer components than illustrated. Each component may include additional, different, or fewer components. The logic illustrated in the flow diagram may include additional, different, or fewer operations than illustrated. The operations illustrated may be performed in an order different than illustrated.

FIG. 8 illustrates a third example of the system 100. The system 100 may include communication interfaces 812, input interfaces 828 and/or system circuitry 814. The system circuitry 814 may include a processor 816 or multiple processors. Alternatively or in addition, the system circuitry 814 may include memory 820.

The processor 816 may be in communication with the memory 820. In some examples, the processor 816 may also be in communication with additional elements, such as the communication interfaces 812, the input interfaces 828, and/or the user interface 818. Examples of the processor 816 may include a general processor, a central processing unit, logical CPUs/arrays, a microcontroller, a server, an application specific integrated circuit (ASIC), a digital signal processor, a field programmable gate array (FPGA), and/or a digital circuit, analog circuit, graphics processing units (GPUs), or some combination thereof.

The processor 816 may be one or more devices operable to execute logic. The logic may include computer executable instructions or computer code stored in the memory 820 or in other memory that when executed by the processor 816, cause the processor 816 to perform the operations of the mentoring framework 104, procedure tracking controller 108, the performance tracking controller 110, the performance enhancement controller 110, the augmented reality controller 114, the image feature recognition framework 118, the gesture recognition framework 120, the medical instruction database 122, procedure model 124, the performance model 126, enhancement model 128 and/or the system 100. The computer code may include instructions executable with the processor 816.

The memory 820 may be any device for storing and retrieving data or any combination thereof. The memory 820 may include non-volatile and/or volatile memory, such as a random-access memory (RAM), a read-only memory (ROM), an erasable programmable read-only memory (EPROM), or flash memory. Alternatively or in addition, the memory 820 may include an optical, magnetic (hard-drive), solid-state drive or any other form of data storage device. The memory 820 may include at least one of the mentoring framework 104, procedure tracking controller 108, the performance tracking controller 110, the performance enhancement controller 110, the augmented reality controller 114, the image feature recognition framework 118, the gesture recognition framework 120, the medical instruction database 122, procedure model 124, performance model 126, enhancement model 128 and/or the system 100. Alternatively, or in addition, the memory may include any other component or sub-component of the system 100 described herein.

The user interface 818 may include any interface for displaying graphical information. The system circuitry 814 and/or the communications interface(s) 812 may communicate signals or commands to the user interface 818 that cause the user interface to display graphical information. Alternatively or in addition, the user interface 818 may be remote to the system 100 and the system circuitry 814 and/or communication interface(s) may communicate instructions, such as HTML, to the user interface to cause the user interface to display, compile, and/or render information content. In some examples, the content displayed by the user interface 818 may be interactive or responsive to user input. For example, the user interface 818 may communicate signals, messages, and/or information back to the communications interface 812 or system circuitry 814.

The system 100 may be implemented in many ways. In some examples, the system 100 may be implemented with one or more logical components. For example, the logical components of the system 100 may be hardware or a combination of hardware and software. The logical components may include the mentoring framework 104, procedure tracking controller 108, the performance tracking controller 110, the performance enhancement controller 110, the augmented reality controller 114, the image feature recognition framework 118, the gesture recognition framework 120, the medical instruction database 122, procedure model 124, performance model 126, enhancement model 128, and/or any component or subcomponent of the system 100. In some examples, each logic component may include an application specific integrated circuit (ASIC), a Field Programmable Gate Array (FPGA), a digital logic circuit, an analog circuit, a combination of discrete circuits, gates, or any other type of hardware or combination thereof. Alternatively or in addition, each component may include memory hardware, such as a portion of the memory 820, for example, that comprises instructions executable with the processor 816 or other processor to implement one or more of the features of the logical components. When any one of the logical components includes the portion of the memory that comprises instructions executable with the processor 816, the component may or may not include the processor 816. In some examples, each logical component may just be the portion of the memory 820 or other physical memory that comprises instructions executable with the processor 816, or other processor(s), to implement the features of the corresponding component without the component including any other hardware. Because each component includes at least some hardware even when the included hardware comprises software, each component may be interchangeably referred to as a hardware component.

Some features are shown stored in a computer readable storage medium (for example, as logic implemented as computer executable instructions or as data structures in memory). All or part of the system and its logic and data structures may be stored on, distributed across, or read from one or more types of computer readable storage media. Examples of the computer readable storage medium may include a hard disk, a floppy disk, a CD-ROM, a flash drive, a cache, volatile memory, non-volatile memory, RAM, flash memory, or any other type of computer readable storage medium or storage media. The computer readable storage medium may include any type of non-transitory computer readable medium, such as a CD-ROM, a volatile memory, a non-volatile memory, ROM, RAM, or any other suitable storage device.

The processing capability of the system may be distributed among multiple entities, such as among multiple processors and memories, optionally including multiple distributed processing systems. Parameters, databases, and other data structures may be separately stored and managed, may be incorporated into a single memory or database, may be logically and physically organized in many different ways, and may implemented with different types of data structures such as linked lists, hash tables, or implicit storage mechanisms. Logic, such as programs or circuitry, may be combined or split among multiple programs, distributed across several memories and processors, and may be implemented in a library, such as a shared library (for example, a dynamic link library (DLL).

All of the discussion, regardless of the particular implementation described, is illustrative in nature, rather than limiting. For example, although selected aspects, features, or components of the implementations are depicted as being stored in memory(s), all or part of the system or systems may be stored on, distributed across, or read from other computer readable storage media, for example, secondary storage devices such as hard disks, flash memory drives, floppy disks, and CD-ROMs. Moreover, the various logical units, circuitry and screen display functionality is but one example of such functionality and any other configurations encompassing similar functionality are possible.

The respective logic, software or instructions for implementing the processes, methods and/or techniques discussed above may be provided on computer readable storage media. The functions, acts or tasks illustrated in the figures or described herein may be executed in response to one or more sets of logic or instructions stored in or on computer readable media. The functions, acts or tasks are independent of the particular type of instructions set, storage media, processor or processing strategy and may be performed by software, hardware, integrated circuits, firmware, micro code and the like, operating alone or in combination. Likewise, processing strategies may include multiprocessing, multitasking, parallel processing and the like. In one example, the instructions are stored on a removable media device for reading by local or remote systems. In other examples, the logic or instructions are stored in a remote location for transfer through a computer network or over telephone lines. In yet other examples, the logic or instructions are stored within a given computer and/or central processing unit (“CPU”).

Furthermore, although specific components are described above, methods, systems, and articles of manufacture described herein may include additional, fewer, or different components. For example, a processor may be implemented as a microprocessor, microcontroller, application specific integrated circuit (ASIC), discrete logic, or a combination of other type of circuits or logic. Similarly, memories may be DRAM, SRAM, Flash or any other type of memory. Flags, data, databases, tables, entities, and other data structures may be separately stored and managed, may be incorporated into a single memory or database, may be distributed, or may be logically and physically organized in many different ways. The components may operate independently or be part of a same apparatus executing a same program or different programs. The components may be resident on separate hardware, such as separate removable circuit boards, or share common hardware, such as a same memory and processor for implementing instructions from the memory. Programs may be parts of a single program, separate programs, or distributed across several memories and processors.

A second action may be said to be “in response to” a first action independent of whether the second action results directly or indirectly from the first action. The second action may occur at a substantially later time than the first action and still be in response to the first action. Similarly, the second action may be said to be in response to the first action even if intervening actions take place between the first action and the second action, and even if one or more of the intervening actions directly cause the second action to be performed. For example, a second action may be in response to a first action if the first action sets a flag and a third action later initiates the second action whenever the flag is set.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

1. A system for comprising:
a processor, the processor configured to:
acquire image data from a camera targeting a location where healthcare is administered;
receive sensor data from a sensor attached to a care provider during performance of a multi-step medical procedure;
generate gesture features based on the sensor data;
generate image features based on the image data;
determine, based on the image features and a machine learning model, a step identifier for a step in a multi-step surgical procedure;
access natural language text descriptive of an instruction to perform the step;
display the natural language text associated with the step on a display accessible to the care provider;
determine, based on the gesture information and a second machine learning model, a performance metric that measures performance of the care provider; and
output the performance metric, wherein to output the performance metric the processor is configured to:
store the performance metric in a memory, communicate the performance metric over a communications network, display the performance metric on the display, or a combination thereof.

2. The system of claim 1, wherein the processor is further configured to:
playback an audible explanation of description of how to perform the step.

3. The system of claim 1, wherein the processor is further configured to:
communicate a live video feed of the medical procedure to a device at a remote location, the device configured to receive input information provided by a remote user remotely viewing the medical procedure, the input information comprising audio, images, text, lines, or a combination thereof.

4. The system of claim 1, wherein the processor is further configured to:
communicate the natural language text to an augmented reality device viewable by the care provider to display input information provided by the remote user.

5. The system of claim 1, wherein the processor is further configured to:
determine respective performance metrics for the steps in the multi-step procedure; and
determine, based on aggregation of the respective performance metrics, a combined performance metric for the multi-step procedure.

6. The system of claim 1, wherein the sensor data comprises a measurement of directional movement information, rotation information, vibration information, position information, or a combination thereof, corresponding to the care provider, or an appendage of the care provider, operating in the location where healthcare is administered.

7. The system of claim 1, wherein the processor is further configured to:
determine the performance metric satisfies an intervention criterion; and
establish, in response to satisfaction of the intervention criterion, communication with a remote node at a different geographic location from where healthcare is administered.

8. The system of claim 7, further comprising:
transmit the performance metric, the image data, the gesture data, or a combination thereof to the remote node.

9. The system of claim 1, the processor is further configured to:
determine the performance metric satisfies an intervention criterion;
in response to satisfaction of the intervention criterion:
acquire a second natural language text specifying an instruction to improve the performance of the step; and
display the second natural language text and/or playback the natural language text in audio format.

10. The system of claim 1, wherein to display the natural language text, the processor is further configured to:
display the natural language text and live video of the location where healthcare is administered on a display device viewable by the care provider.

11. A method, comprising:
acquiring image data from a camera targeting a location where healthcare is administered;
receiving sensor data from a sensor attached to a care provider during performance of a multi-step medical procedure;
generating gesture features based on the sensor data;
generating image features based on the image data;
determining, based on the image features and a machine learning model, a step identifier for a step in a multi-step surgical procedure;
accessing natural language text descriptive of an instruction to perform the step;
displaying the natural language text associated with the step on a display accessible to the care provider;
determining, based on the gesture information and a second machine learning model, a performance metric that measures performance of the care provider; and
outputting the performance metric by storing the performance metric in a memory, communicating the performance metric over a communications network, displaying the performance metric on the display, or a combination thereof.

12. The method of claim 11, further comprising:
playing back an audible explanation of description of how to perform the step.

13. The method of claim 11, further comprising:
transmitting a live video feed of the medical procedure to a device at a remote location, the device configured to receive input information provided by a remote user remotely viewing the medical procedure, the input information comprising audio, images, text, lines, or a combination thereof.

14. The method of claim 11, further comprising:
transmitting the natural language text to an augmented reality device viewable by the care provider to display input information provided by the remote user.

15. The method of claim 11, further comprising:
determining respective performance metrics for the steps in the multi-step procedure; and
determining, based on aggregation of the respective performance metrics, a combined performance metric for the multi-step procedure.

16. The method of claim 11, wherein the sensor data comprises a measurement of directional movement information, rotation information, vibration information, position information, or a combination thereof, corresponding to the care provider, or an appendage of the care provider, operating in the location where healthcare is administered.

17. The method of claim 11, further comprising:
determine the performance metric satisfies an intervention criterion; and
establish, in response to satisfaction of the intervention criterion, communication with a remote node at a different geographic location from where healthcare is administered.

18. The method of claim 17, further comprising:
transmitting the performance metric, the image data, the gesture data, or a combination thereof to the remote node.

19. The method of claim 11, further comprising:
determining the performance metric satisfies an intervention criterion;
in response to satisfaction of the intervention criterion:
acquiring a second natural language text specifying an instruction to improve the performance of the step; and
displaying the second natural language text.

20. The method of claim 11, wherein displaying the natural language text further comprises:
displaying the natural language text and live video of the location where healthcare is administered on a display device viewable by the care provider. 21-32. (canceled)
